(19) United States
(12) Patent Application Publication
(10) Pub. No.: US 2006/0095000 A1
(43) Pub. Date: May 4, 2006

(54) Sealed container assemblies having readily fracturable opener seams

(76) Inventor: Steven D. Kimmell, Granada Hills, CA (US)

Correspondence Address: Patent Counsel;Robert W. Mulcahy, MS/2061, 520 Sequoia Drive, Sunnyvale, CA, 94086, US

(21) Appl. No.: 11/259,007

(22) Filed: Oct. 26, 2005

Related U.S. Application Data
(60) Provisional application No. 60/623,465, filed on Oct. 29, 2004.

(51) Int. Cl. A61M 35/00

(52) U.S. Cl. 604/1

(57) ABSTRACT

A closed plastic container has a plastic elongated sleeve which has a hollow interior. A line of embrittlement is inscribed by a laser within the thickness of the sleeve wall to form a breaking plane seam for opening the container. The container is opened by squeezing or twisting the container at the scribed line seam. The closed container may have an applicator material inside or outside of the container. A substance contained within the container is dispensed and applied when the container is opened at the inscribed plastic embrittlement line. The preferred plastic container material is poly(ethylene terephthalate).

This application claims benefit of U.S. Provisional Application No. 60/623,465, filed Oct. 29, 2004, which is hereby incorporated by reference in its entirety.

### BACKGROUND OF THE INVENTION

1. Field of the Invention

The present invention generally relates to substance applicator containers, and more specifically the present invention relates to closed sealable assembly containers which have a fracturable (fracture) plane on the exterior surface for the purpose of opening the containers. The closed containers may contain a substance, such as a medical fluid or a chemical or radioactive, which is dispensed and applied when the container is opened at the fracture plane. The present invention also relates to methods of making the containers and methods of using the containers.

2. Description of the Related Art

Swab applicators are one example of a product which has a container. Swab applicators typically include a swab having a cotton or other absorbent or porous material applicator head connected to a handle. The swab is contained within a closed container package which can be opened to expose the swab for widespread use for medicinal, cleaning and cosmetic substances, generally liquids. For example, the swab may contain a medical fluid which can be applied to a patient. A demand exists for self-contained sealable units consisting of a swab prepackaged with the substance for which it is to be used, due to the convenience and sanitation benefits that are afforded thereby.

One example of a swab applicator assembly is U.S. Pat. No. 6,516,947, issued Feb. 11, 2003 and hereby incorporated by reference in its entirety, directed to a swab container having a fracture recess on the exterior of the container assembly to allegedly enable facile and reliable opening of the container. The fracture recess extends from the exterior surface of the container wall into the wall whereby the wall has a reduced wall thickness at the recess, facilitating reliable fracture and breakage at that portion of the sealed container. The swab container or package is opened by squeezing or bending at the exterior recess portion of the thinner wall portion of the package to break open the container and expose the swab. While this container is effective in providing a predictable and reliable breaking point for the swab package, there is always the problem of potential unclean breakage because of an imprecise and non-uniform recess and the required application of force for opening the package.

Another example of a swab applicator can be found in U.S. Pat. No. 4,952,204, hereby incorporated by reference in its entirety, entitled Dry Handle Swab Assembly Unit, which issued on Aug. 28, 1990. The '204 patent describes a swab contained within a sleeve which can readily be opened by use of manual force. The swab has a substance which can be applied by the swab. The swab has a straight hollow plastic stick with a bud of cotton attached on one end. The sleeve consists of a relatively small diameter cylindrical handle portion at one end, a substantially larger diameter receptacle portion at the opposite end, and a transition portion of compound configuration there between. A tip of the handle portion of the sleeve engages a tip of the swab stick. The intersection between the receptacle and transition portions forms a sharp angle on an interior of the sleeve. The sleeve is opened by squeezing the sleeve at the intersection between the receptacle and transition portions and breaking the sleeve at the interior sharp angle.

Examples of other containers include applicators or dispensers having sharp interior angles to open the containers, and include U.S. Pat. No. 4,927,012 entitled Packaging Assembly for Substances to be Post-Mixed, which issued on May 22, 1990, U.S. Pat. No. 5,229,061 entitled Mold and Method for Producing a Hollow Tube Component for a Dispensing Applicator, which issued on Jul. 20, 1993, and U.S. Pat. No. 5,326,603 entitled Hollow Tube Component for a Dispensing Applicator, which issued on Jul. 5, 1994.

Existing applicators and dispensers can be improved. For example, existing applicators and dispensers having recesses require difficult tolerances for clean breakage at the recess and those swab packages utilizing sharp interior angles for opening the applicators and dispensers have a relatively complex structure. Molds having intricate shapes or critical preparation methods are required in order to manufacture such applicators and dispensers. Because of the intricate structure of the interior sharp angles which is used for opening the applicators and dispensers, there are limited locations on the applicators and dispensers where the sharp interior angle can be formed. Also, the exterior shape of the applicators and dispensers tend to be more intricate in the area of the interior sharp angle.

### SUMMARY OF THE INVENTION

The present invention is based on the discovery of means, materials and techniques for laser applied and sectional controlled embrittling of plastic wall container having certain resin compositions. Specifically, it has been found that the focused application of laser radiation to certain resin surfaces, such as poly(ethylene terephthalate) (PET), results in a controlled localized molecular disarrangement so as to cause embrittlement in, and facile breakage of, the treated resin. The application of laser radiation circumferentially to a cylindrical thin walled container results in a defined circumscribed embrittlement zone or plane which defines a facile breakage point in the container.

The present invention provides new containers which have a new structure for opening the containers and methods for their preparation. The new containers have a line circumscribing the container which is made brittle and frangible by the application of laser energy. The container itself is simple plastic tubing which is manufactured on low-cost extrusion tooling and is widely available from stock. The closing and sealing of the container is accomplished by simple and low cost heat-sealing equipment which is also widely available. Due to the simplicity and regularity of the basic enclosure structure, being a cylinder, marking, and labeling may be readily applied prior to filling and sealing on continuous printing and marking equipment. Examples of the present invention include applicators and dispensers in general. and more specifically, swab applicators and pop (or snap) ampule packages.

One plastic container configuration according to the present invention has a plastic elongated sleeve having a hollow interior, the sleeve creating a wall of certain thickness. A line of embrittlement is created in the wall's exterior extending into the interior of the sleeve by exposure of the wall surface to a laser which disrupts the physical plastic structure of the wall. The embrittlement line extends about the entire periphery of the wall surface so as to form a connected circumferential plane about the surface of the sleeve potion of the container. This may cause an embrittlement plane of indeterminate shape dependent upon the material, the type of laser, the rate of exposure, the energy of the laser, and any intervening lens(es) used. The embrittlement line can be of any predetermined shape, eg. V-shape, U-shaped, linear shaped etc. As indicated above, poly (ethylene terephthalate) is the preferred resins for the containers presented herein.

One swab applicator according to the present invention has a closed plastic container package having a portion of the tubular enclosure compressed under heat and pressure to engulf and co-melt the handle of a swab enclosed in the interior of the closed package, thus attaching the swab to the tubular enclosure and forming a seal around and inclusive of the swab handle. In accordance with the present invention, a circumscribed embrittlement plane line is formed about the tubular plastic container, at approximately the mid-line of the total length of the package, so as to form a fracturable plane for opening the container. The container is opened by squeezing, bending or twisting the container at the scribed line seam.

A preferred embodiment of the present invention is the provision of a sealable assembly comprising a swab having an elongated, small diameter stick with an applicator element at one end thereof, and an elongated, thin-wall hollow sleeve assembled with the swab. The sleeve is integrally formed as a single piece container, from a relatively rigid plastic material that is manually compressible and breakable, and the container has a handle portion at one end, a receptacle portion at the other end, and a transition portion there-between in the form of the circumscribed laser induced embrittlement described herein. The handle portion, which includes a stick applicator holder element that engages the handle, extends along a major part of its length. It conforms generally to the stick, but is spaced slightly from the surface thereof throughout most of the coextensive length, to provide sufficient clearance for facile assembly while minimizing the gap therebetween. The cross section of the receptacle portion is the same as that of the sleeve at the embrittlement plane intersection between the receptacle, this transition portion being so configured that compression or applied force of the sleeve thereat will create a significant level of stress, thereby facilitating manual severance of the sleeve at the frangible embrittlement plane of the intersection. Generally, because of the relative lengths of the stick and the handle portion, the applicator element of the swab will be contained, at least substantially, within the receptacle portion of the sleeve.

Generally, the receptacle and sleeve portions of the container will be cylindrical in shape and will be generally be equal in diameter at the embrittlement intersection portion; a short stick tip component at the end of the handle portion will advantageously provide the stick-engaging element. Typically, the swab will be about 11 centimeters in length; the sleeve handle portion will be about 6 centimeters long, and the receptacle portion and the adjacent frangible embrittlement transition portion thereof will have diameters of about 1.15 to 1.30 centimeters. When the plastic material employed for fabrication of the sleeve is polypropylene, it will desirably have a substantially uniform thickness of approximately 0.3 millimeter.

As indicated the instant readily openable container assembly is prepared by impinging the wall of a plastic element or container with a focused energy source that will disrupt the molecular arrangement of the plastic composition and create a line or a plane of embrittlement through the wall of the plastic element and render that embrittlement zone frangible and easily breakable or openable at that line or zone of demarcation. A method of making a fracturable plastic container is provided herein and comprises (i) providing an enclosed hollow plastic container secured to a rotatable mounting element; (ii) placing the mounted container assembly in front of a source of electromagnetic beam radiation energy; (iii) actuating the radiation source to form a radiation beam and simultaneously rotating the mounted container whereby the radiation impinges the surface of the plastic container and causes a molecularly disrupted embrittlement plane extending through the impinged wall of the container.

Other advantages of the invention are attained by the provision of a swab or applicator unit, including an applicator assembly as hereinabove described, and a contained substance. The substance will normally be a liquid, and it will be confined substantially within the receptacle and transition portions of the sleeve, the latter being closed at the free end of its receptacle portion for that purpose.

### BRIEF DESCRIPTION OF THE DRAWINGS

FIG. 1 is a perspective view of a self-contained swab unit embodying the present invention, the enclosed swab being shown in dotted line;

FIG. 2 is an exploded perspective view showing the swab and sleeve of the assembly of which the unit of FIG. 1 is comprised;

FIG. 3 is an elevational view of the fractured unit of FIG. 1, with the integral sleeve and swab shown in section;

FIG. 4 are perspective views of a mandrel used in laser processing of the instant applicator containers;

FIG. 5 demonstrates the application of laser to form the fracturable applicator containers of the present invention; and

FIG. 6 demonstrates detail of the application of laser in FIG. 5 to form the fracturable applicator containers of the present invention.

FIG. 7 is a perspective view of a molded ampule configuration of a self contained swab unit embodying the present invention.

### DETAILED DESCRIPTION OF THE INVENTION

Although the present invention can be made in many different forms, the presently preferred embodiments are described in the disclosure and shown in the attached drawings. This disclosure exemplifies the principles of the present invention and does not limit the broad aspects of the invention only to the illustrated embodiments.

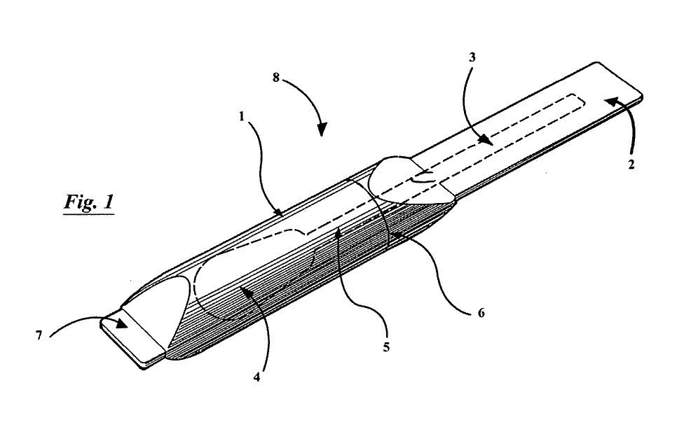

The present invention generally pertains to plastic containers having a brittle line for opening the containers. A specific example of the present invention is shown and described, which is an applicator/dispenser. A new swab applicator according to the principles of the present invention is shown by way of example in FIGS. 1, 2, and 3. The swab applicator 8, has a swab 4 contained within a sealed package 1. The package is sealed closed and provides a container to contain the swab 4. The package 1 has a package handle 2 at one end and a swab head enclosure seal 7 at the opposite end.

Preferably, the package 1 has a generally elongated cylindrical shape. The package handle is formed integrally with the cylindrical body of the package by subjecting a portion of the cylinder to heat and pressure, deforming and flattening it, and trapping the swab handle 5 and co-melting and sealing it 3 into the package handle 2. The swab 4 is contained inside of the package 1, and has a swab handle 5 and a swab head 4 at one end of the swab handle 5. The swab head 4 may be made from a rayon, cotton or foam material or any other suitable material. The swab handle 5 has a proximal end 3 which is engaged with the package handle 2 by means of being sealed together by heat and pressure. A gap may be provided between the package handle 2 and the swab head. The swab handle 5 has a length such that the swab head 4 is contained within the un-compressed portion of the package.

The swab applicator package 1, has a sealed end 7, opposite the end of the package handle 2. The sealed ends 7 and 2 may be sealed by various mechanisms including heat and pressure and ultrasonic welding. Accordingly, the swab head 4 and handle 5 are contained within an interior of the sealed package 1. The swab head 4, may contain various substances. For example, the swab head 4 may contain a medical fluid which can be applied to a patient during use of the swab applicator. The swab applicator can be used for purposes other than medical applications. For example the swab applicator could be used for application of cosmetics. Although the present invention is described as an applicator, a substance does not have to actually be applied by the swab 4 to practice the invention. For example the swab applicator can be used without a substance on the swab head 4 by using the swab to collect a sample from another source.

The swab applicator has a line of embrittlement circumscribing the package at 6. The application of electromagnetic energy, generally in the form of laser energy, to the material along this line creates a weak plane in the structure of the package. This preferably completely circumscribes the package 1, although it may extend around only a portion of the package. The swab applicator 8 is used by breaking the package 1 along the brittle line 6. This is accomplished by bending, twisting or squeezing the package at this point, and thus creating localized stress which fractures the package.

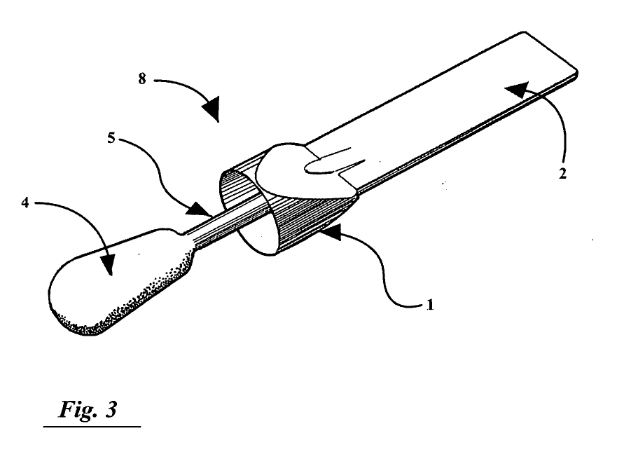

Referring to FIG. 3, a portion of the enclosure is removed from the remainder of the package, exposing the swab head for use. Any superfluous fluid will remain with the removed portion of the package to be discarded. The form of the swab applicator 8 is such that the hand of the user is protected from contact with the fluid and remains dry. At the same time, sufficient support is given to the swab handle 5 by the fused package handle to insure stability and control in use.

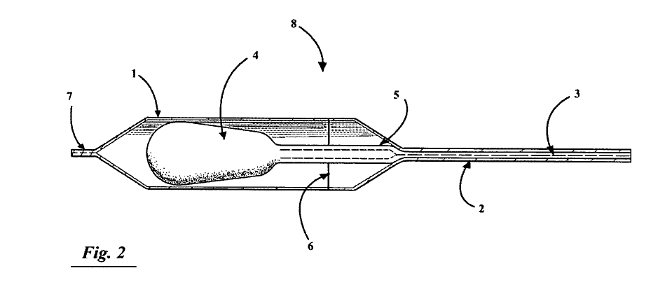

Referring to FIG. 2 the interior diameter of the swab enclosure along its longitudinal length is shown as being constant, assuring consistent control and minimizing any wiping of the swab head against the smooth internal surface when opening the applicator for use.

For best results, it has been found that a sleeve having dimensions such as those typified hereinabove will be fabricated from poly(ethylene terephthalate), in a thickness of about 0.3 millimeter. This will afford a level of rigidity that will provide good handling and structural features while, at the same time, tending to produce fracture upon manual compression or applied stress at the frangible embrittlement joint.

It must of course be appreciated that, if the plastic used for the instant invention is excessively rigid and brittle, the possibility of inadvertent fracture will exist. Furthermore, the resin must have a sufficiently low melt viscosity to permit coverage of all mold surfaces, and it must produce a nonporous and pinhole-free structure. Within the foregoing constraints, any of a variety of synthetic resinous materials may be utilized, and the selection thereof will be evident to those skilled in the art.

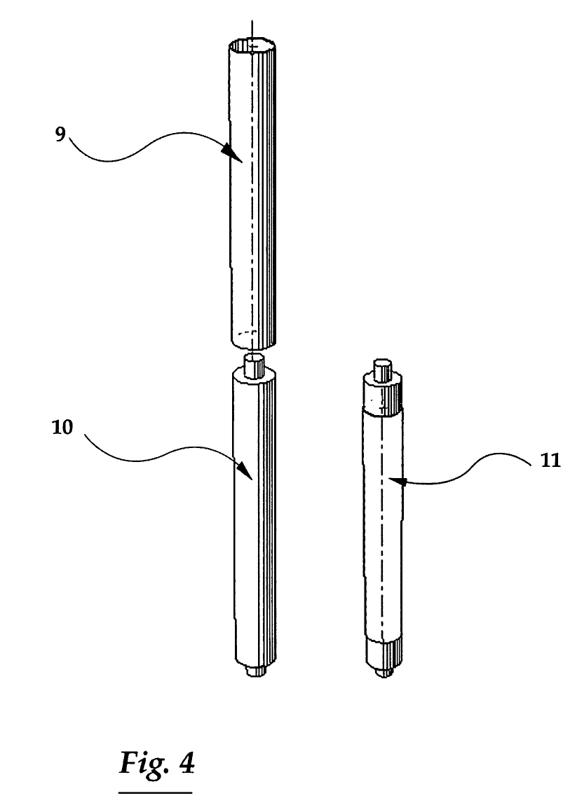

Referring to FIG. 4, the process for imposing the laser energy upon an un-sealed tube 9, is shown. The tube, 9, is slid concentrically onto a mandrel, 10 with a sliding but intimate fit as shown in legend 11, which intimacy assures the circularity of the tube.

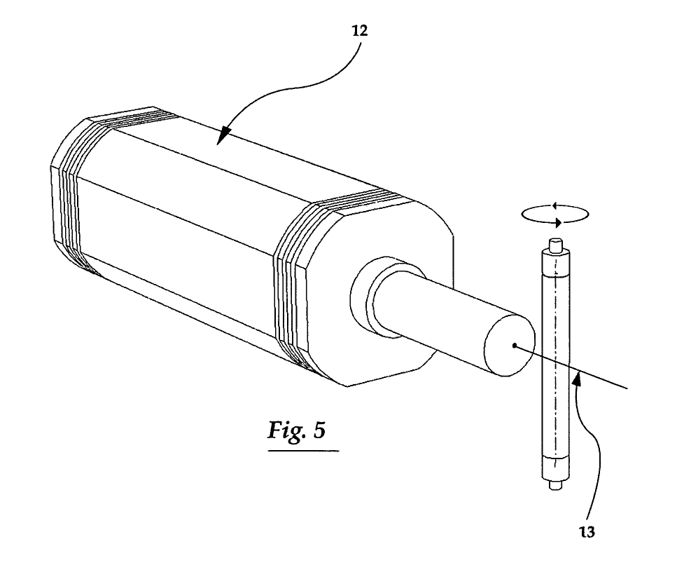

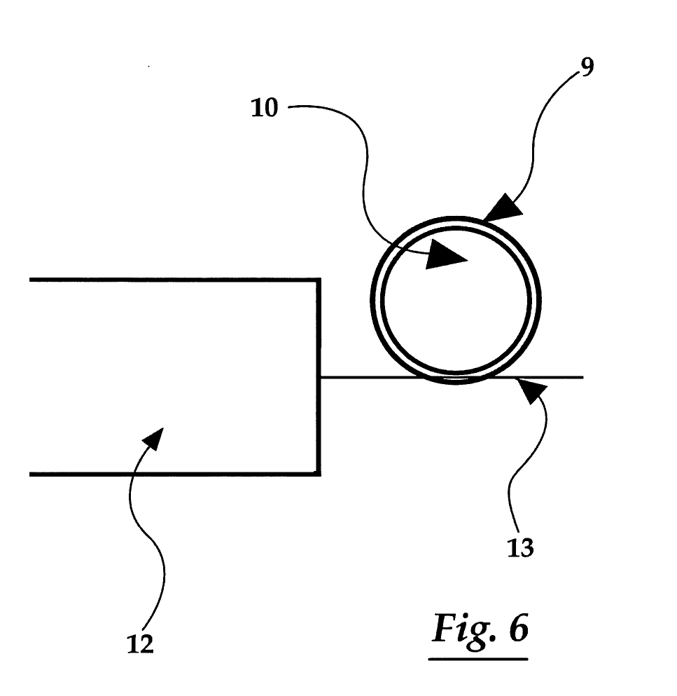

Referring to FIG. 5, the mandrel is position in front of a 40 watt 60-1 Synrad laser shown as 12, manufactured by Synrad, Inc of Mulkilteo, Wash., with a 2.5-inch positive meniscus lens, which provides a spot size of 0.004-inches and a depth of focus of 0.07 inches. The operation of the Synrad equipment is disclosed and published in an operations manual available on the website www.synrad.com, said manual being hereby incorporated by reference. The mandrel is so positioned that the beam emitted by the laser, 13, enters the wall of the tube, 9, tangentially to the circular cross section. This is further illustrated in FIG. 6 where the beam emanating from the laser equipment 12 tangentially impacts the surface of tube 9 housed on the rotating mandrel 10.

The process of embrittlement is carried out by activation of the laser for about 00.14 seconds in an atmosphere containing Nitrogen at 10 pounds per square inch as an assist gas. A pulse of 140 microseconds at a frequency of 3.5 hertz and three revolutions of the mandrel at 1320 rpm completes the process. As an alternative to Nitrogen, 5 psi of clean filtered air, can be used as an assist gas. While the Synrad equipment and parameters are demonstrated herein, any equipment and laser applications known to those skilled in the art may be employed.

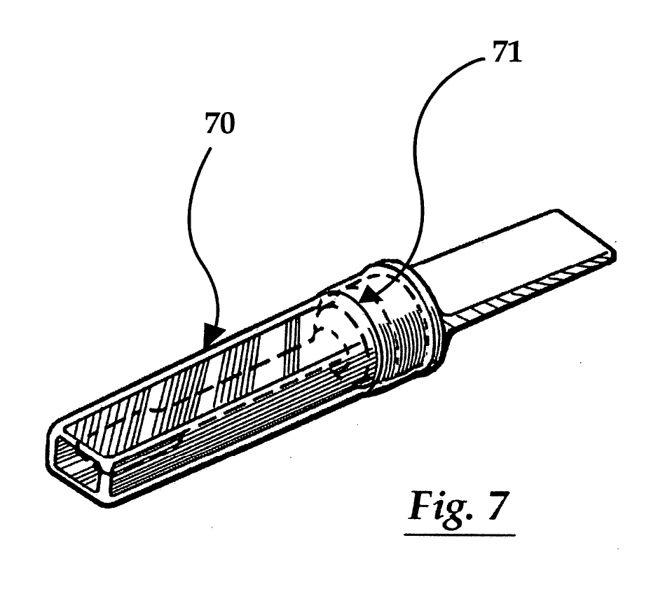

Turning to FIG. 7 there is demonstrated an ampule 70 which is a container with a body and a stem with a neck in between which can be readily fractured. The ampule here has a body portion comprised of poly(ethylene terephthalate) which has a circumscribed plane of embrittlement 71 prepared by laser enscription in the same or sililiar manner described herein for other containers. The ampule is readily fractured at the embrittlement zone 71.

Having thus described the principals of the invention, together with illustrative embodiments thereof, it is to be understood that although specific terms are employed, they are used in a generic and descriptive sense and not for the purpose of limitation, the scope of the invention being set forth in the following claims.

What is claimed:

1. A manually openable plastic container assembly having an enclosed area defined by at least one wall comprising an electromagnetic radiation beam enscribed fracturable plane of frangible embrittlement in the wall to enable ready fracture and opening of the container.

2. The plastic container of claim 1 wherein the container wall is spherical in shape.

3. The plastic container of claim 1 wherein the container wall is cylindrical in shape.

4. The plastic container of claim 1 wherein the frangible embrittlement enscription is engendered by laser radiation.

5. The plastic container of claim 1 wherein the plastic material is poly(ethylene terephthlate).

6. The plastic container of claim 3 wherein the cylindrical shaped container is in the form of a closed tubular component sealed at both ends by heat and pressure; and further comprises:
(i) a swab contained within the closed interior, the swab having a handle connected to one end of the tubular portion by sealing with heat and pressure and a head located within the tubular component; and
(ii) the laser-inscribed line of embrittlement in the wall of the tubular component being located at the approximate mid-point of the tubular component, or at a point where the tubular component has a substantially circular cross-section.

7. The plastic container of claim 1, wherein the laser-embrittled inscription circumscribes the tubular component.

8. A sealable, manually openable plastic container assembly comprising:
i) a plastic elongated sleeve defining a hollow thin wall interior of certain diameter which is reduced in dimension at a first end handle portion of the sleeve relative to an opposite end sleeve portion;
ii) a substance applicator in the hollow interior secured to the first end handle portion of the sleeve;
iii) a substance receptacle portion connected to the other end of the sleeve; and
iv) a transition zone at the intersection between the handle and receptacle sleeve ends, the zone comprising a plane of laser induced fracturable embrittlement inscribed in the wall about the container to facilitate manual opening by application of stress at the plane of embrittlement.

9. The plastic container of claim 7, wherein the laser-embrittled inscription circumscribes the elongated sleeve.

10. The plastic container of claim 8 wherein the elongated sleeve has a substantially cylindrical shape, and the inside diameter of the elongated sleeve is substantially constant from a location above the laser-embrittled inscription to a location below the laser-embrittled inscription.

11. The plastic container of claim 9, wherein the substance applicator is a swab head connected to a thin stick swab handle.

12. The plastic container of claim 10 wherein (i) the elongated sleeve and swap applicator is in the form of a closed package having a substantially constant cylindrical shape, with one end being a material receptacle and sealed under heat and pressure, and the other end including the handle of the swab sealed and flattened under heat and pressure; and (ii) the circumscribed line of laser embrittlement imposed on the wall of the closed package being located at a position on the enclosure spaced away from the end which is sealed including the handle of the swab.

13. The swab applicator of claim 11, wherein the swab head enclosure has an interior diameter of the swab head enclosure is substantially constant from a location above the laser embrittlement line to a location below the laser embrittlement line.

14. The applicator package of claim 12, wherein the closed package has an elongated tubular shape and the laser embrittlement circumscribes at least a portion of the exterior surface of the closed package such that the interior is exposed for use when the closed package is opened along the laser embrittlement.

15. A method of making a fracturable plastic container comprising:
i) providing an enclosed hollow plastic container secured to a rotatable mounting element;
ii) placing the mounted container assembly in front of a source of electromagnetic beam radiation energy;
iii) actuating the radiation source to form a radiation beam and simultaneously rotating the mounted container whereby the radiation impinges the surface of the plastic container and causes a molecularly disrupted embrittlement plane extending through the impinged wall of the container.

16. The method of claim 14 wherein the container is tubular and mounted on a rotatable mandrel.

17. The method of claim 15 wherein the electromagnetic radiation is in the form of a laser.

18. The method of claim 16 wherein the plastic container material is poly(ethylene terephthalate.

19. The method of claim 17 wherein the laser is applied at a frequency of 3.5 Hertz and a pulse of about 140 microseconds.

20. The method of claim 19 wherein the mandrel is rotated at about 1320 rpm.
